(19) United States
(12) Patent Application Publication
(10) Pub. No.: US 2019/0338051 A1
(43) Pub. Date: Nov. 7, 2019

(54) PREPARATION METHOD FOR AND USE OF REDOX-RESPONSIVE CHITOSAN-LIPOSOME

(71) Applicant: DALIAN MINZU UNIVERSITY, Dalian (CN)

(72) Inventors: Shubiao ZHANG, Dalian (CN); Huiying CHEN, Dalian (CN); Yu MA, Dalian (CN); Xiaoli QIN, Dalian (CN); Haoming LAN, Dalian (CN); Shaohui CUI, Dalian (CN)

(21) Appl. No.: 16/466,362

(22) PCT Filed: Dec. 4, 2017
(86) PCT No.: PCT/CN2017/114413

§ 371 Date: Jun. 4, 2019

(30) Foreign Application Priority Data
Jul. 27, 2017 (CN) 201710620705.7

(51) Int. Cl. C08B 37/08; A61K 9/00; A61K 9/127; A61K 49/18; A61K 41/00; C12N 15/88

(52) U.S. Cl. CPC C08B 37/003; A61K 9/0009; C12N 15/88; A61K 49/1812; A61K 41/00; A61K 9/1271

(57) ABSTRACT

The present invention provides a preparation method of a redox-responsive chitosan-liposome and use thereof, wherein the method uses dithiobis succinimidyl-substituted ester to synthesize a redox-responsive and disulphide bonded double fatty chain substituent phosphatidylethanolamine-s-s-chitosan. Using the synthesized double fatty chain substituent phosphatidylethanolamine chitosan, by a post-insertion and self-assembly method, to modify liposome, to construct a double fatty chain substituent phosphatidylethanolamine chitosan-liposome drug carrier having a redox-responsive chitosan brush on the surface thereof. The chitosan-liposome constructed in the present invention not only has the strong cell adhesion property and the antiserum property, but also has environmental response properties, being suitable for the intravenous injection. The present invention also provides the use of the chitosan-liposome encapsulating super-paramagnetic ferroferric oxide nanoparticles in drug delivery, which has high drug delivery efficiency and high biocompatibility, and has broad application prospects.

### FIELD OF THE INVENTION

The present invention relates to a drug carrier of a redox-responsive chitosan-liposome, more particularly to a redox-responsive double fatty chain substituent phosphatidylethanolamine chitosan and a drug carrier encapsulating super-paramagnetic ferroferric oxide nanoparticles constructed the chitosan with liposome, and relates to a method of preparing a new drug carrier belonging to the field of drug delivery.

### BACKGROUND OF THE INVENTION

A drug carrier is a system capable of changing the way a drug enters the human body, and the distribution of the drug in vivo, controlling a release rate of the drug and delivering the drug to the target organ. By controlled release, the drug carrier can effectively improve the utilization rate, safety and effectiveness of the drug. Chitosan and liposome are commonly used drug carriers, chitosan has good biocompatibility and biodegradability, the 2-position amino group and the 6-position hydroxyl group are easily structurally modified, has bioadhesive properties, and capable of improving instantaneous penetration ability of the drug among cells by opening the cell channel; and the liposome are ultrafine spherical particles with a structure of one or more layers of lipid vesicles formed by dispersing an amphiphilic surfactant in water, they can be loaded with water-soluble or liposoluble drugs, and are widely used in drug carriers.

A multifunctional nanocarrier is a new generation of nanocarrier developed based on the single function nanocarrier, it overcomes some shortcomings of the single functional nanocarrier in tumor diagnosis and treatments, such as real-time monitoring of cellular activity in the body, specific targeting of a target site or efficient delivery of a drug within target cells. The multifunctional nanocarrier combines different functions in a single stable structure. For example, the combination of a tumor developer or a diagnostic reagent to achieve early diagnosis of tumors, real-time monitoring of tumor therapeutic effect, and the like. The multifunctional nanocarrier offers new opportunities for the early diagnosis and the individualized drug treatment of tumors.

Magnetic resonance imaging (MRI) has good soft tissue resolution and spatial resolution, which can not only clearly display the anatomic structure of tissue, but also accurately locate and quantitatively analyze the imaging characteristics of the soft tissue, and is one of the most efficient methods for early diagnosis of tumors. In order to enhance the contrast between the image of diseased tissue and normal tissue to improve the clarity of the diseased tissue, a suitable contrast agent is required to display anatomic characteristics. The T2 contrast agent has a higher magnetic moment than the paramagnetic substance, and has a significant acceleration effect on the relaxation of protons in adjacent tissue, which can greatly improve the detection sensitivity. Common superparamagnetic contrast agents are mainly microcrystalline metal particles of different sizes (e.g. Fe₃O₄, γFe₂O₃).

The malignant tumor is the first killer of human health. Although the continuous improvement in the detection and treatments in recent years, the survival rate of cancer patients has increased, the mortality rate of the cancer patients remains substantially high. At present, chemotherapy is one of major tumor treatments, but the toxicity of drug and the resistance of tumor cells lead to a low cure rate of chemotherapy. On the other hand, the lack of effective early diagnosis is also a main cause of the low cure rate. Therefore, seeking for new and efficient early diagnosis and treatments of tumors is an urgent problem to be solved in clinical oncology. Gene therapy repairs defective genes that cause diseases or suppress harmful genes that causes diseases by introducing therapeutic genes into nucleuses of target cells, thereby returning the body to normal function, achieving the goal of treating diseases. A safe and efficient carrier is one of the keys to the success of gene therapy.

Stability of a drug carrier in blood is critical to the function of the drug carrier. The structure of liposome is susceptible to damage by components such as high-density lipoprotein in serum, leading to the leakage of encapsulated drugs. Chitosan has a good antiserum property, assisting in improving the stability of drug-loaded nanoparticles in the serum. The chitosan modifies the liposome by the post-insertion method, so as to construct a drug carrier with a chitosan brush on its surface, and encapsulating nano-ferric oxide nanoparticles (SPIO) to from a multifunctional drug carrier which integrates drug delivery and imaging diagnosis, and uses genes as model drugs to evaluate gene transfection properties, to realize integration of treatments and diagnosis, which opens up a new path for the tumor treatments.

There are significant different redox environments in the extracellular and cellular areas of human lesions (such as tumors), wherein the extracellular environment tends to be an oxidizing environment, in order to maintain the stability of disulphide bonds such as membrane proteins, while the intracellular environment is a reducing environment formed by overexpressed high-concentration glutathione, thus by using the difference in the concentration between extracellular and intracellular redox species, the controlled release of the drugs by the carrier may be achieved, and further to improve the therapeutic effect.

### SUMMARY OF THE INVENTION

The technical problems to be solved by the present invention are to overcome defects of the existing drug carriers, to provide a redox-responsive double fatty chain substituent phosphatidylethanolamine chitosan, and a liposome carrier with a redox-responsive chitosan brush and SPIO constructed by post-insertion, a preparation method of the chitosan-liposome drug carrier.

The first object of the present invention is to request protection of redox-responsive chitosan, having a structure of formula (I):

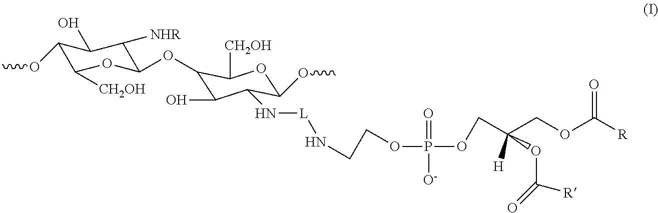

wherein, L=—CO—(CH₂)ₐ—S—S—(CH₂)_{b}—CO—, a=1-5, b=1-5; and R and R′ are identical or different CₓH_{y}, wherein x=11-17, y=21-35.

Preferably, L=—CO—(CH₂)₂—S—S—(CH₂)₂—CO—, R and R′ are identical or different C₁₁H₂₃, C₁₃H₂₇, C₁₇H₃₅ or C₁₇H₃₃.

Another object of the present invention is to request protection of a preparation method of the redox-responsive chitosan, specifically comprising: dissolving chitosan in water firstly, and adding, if necessary, 1-3 glacial acetic acid thereto to fully dissolve the chitosan, under a stirring state, dropwise adding to a DMSO solution of dithiobis succinimidyl-substituted ester, reacting at 20-60° C. for 1-24 h, thereafter continuously adding dropwise an ethanol solution of double fatty chain substituent phosphatidylethanolamine to the reaction solution, reacting at 20-60° C. for 1-24 h, and after rotary evaporation, the reaction solution being subjected to dialysis, lyophilize to prepare the redox-responsive chitosan.

Preferably, a weight average molecular weight of the chitosan is 500-10000 Da, and a degree of deacetylation is 65-95%.

Preferably, the double fatty chain substituent phosphatidylethanolamine is one or more of 1,2-dilauroyl-sn-glycero-3-phosphoethanolamine, 1,2-distearoyl-sn-glycero-3-phosphatidylethanolamine, 1,2-dimyristoyl-sn-glycero-3-phosphatidylethanolamine, 1,2-dipalmitoyl-sn-glycero-3-phosphoethanolamine and 1,2-dioleoyl-sn-glycero-3-phosphoethanolamine, but is not limited to the aforementioned raw materials, its amount is 0.1-1 times a molar equivalent of a repeating unit of the chitosan, preferably 0.3-0.6 times, and a reaction condition is preferably stirring reaction at 20-50° C. for 2-48 h, more preferably stirring reaction at 30-50° C. for 4-12 h.

The third object of the present invention is to provide a preparation method of a redox-responsive chitosan-liposome drug carrier:

Firstly using the film-ultrasonic method to prepare a liposome @ SPIO composite material, then modifying a cationic liposome by means of post-insertion and self-assembly, the redox-responsive chitosan is inserted into the phospholipid bilayer of the liposome, to obtain the redox-responsive chitosan-liposome, wherein a mass ratio of the cationic liposome to SPIO is 30:1-10:1, and a mass ratio of the chitosan to the liposome@SPIO is 0.5:1-6:1.

Preferably, the cationic liposome is any one of DOTAP, Lipofectin and Lipofectamin™ 2000.

Preferably, a particle diameter of the SPIO nanoparticles is 1-30 nm.

The present invention also claims the use of the abovementioned redox-responsive chitosan-liposome as a drug carrier, especially the use in gene transfection.

The present invention uses redox-responsive double fatty chain substituent phosphatidylethanolamine to modify the chitosan, then modifies the liposome @ SPIO by post-insertion, to obtain a double fatty chain substituent phosphatidylethanolamine chitosan-liposome drug carrier with a redox-responsive chitosan brush on its surface, to improve the antiserum ability and biocompatibility of the liposome, and improve the ability to control the release of drugs through the response to the redox environments, and provide magnetic field directed targeting function and magnetic resonance imaging function, to realize integrated drug delivery in therapy and diagnostics.

Compared with the prior art, the present invention has following advantages:

1. The present invention uses redox environment responsive double fatty chain substituent phosphatidylethanolamine to modify the chitosan, to prepare the double fatty chain substituent phosphatidylethanolamine chitosan.

2. The present invention uses the redox environment responsive double fatty chain substituent phosphatidylethanolamine chitosan to modify liposome by post-insertion, to improve the biocompatibility and the blood stability of the liposome, and through environmental responsive properties, it is suitable for the intravenous injection, and improves the ability to control the release of drugs.

3. The present invention uses a chitosan-liposome-encapsulated super-paramagnetic ferroferric oxide nanoparticles to obtain a composite carrier of redox-responsive chitosan-liposome, thereby realizing its use in drug delivery, and especially in gene transfection. Such complex has high drug delivery efficiency and high biocompatibility, and provides magnetic field-directed targeting function and magnetic resonance imaging function, and has broad application prospects.

### BRIEF DESCRIPTION OF THE DRAWINGS

FIG. 1 is a FTIR spectrum of redox-responsive chitosan prepared in example 1;

FIG. 2 is a ¹HNMR spectrum of redox-responsive chitosan prepared in example 1;

FIG. 3 is a TEM photograph of a redox-responsive chitosan-DOTAP liposome-SPIO composite carrier prepared in example 1;

FIG. 4 is a graph showing the retardation ability to DNA of the redox-responsive chitosan-liposome prepared in Example 1;

FIG. 5 shows the gene transfection efficiency of redox-responsive chitosan-liposome prepared in the present invention; and

FIG. 6 shows the cytotoxicity of redox-responsive chitosan-liposome prepared in the present invention.

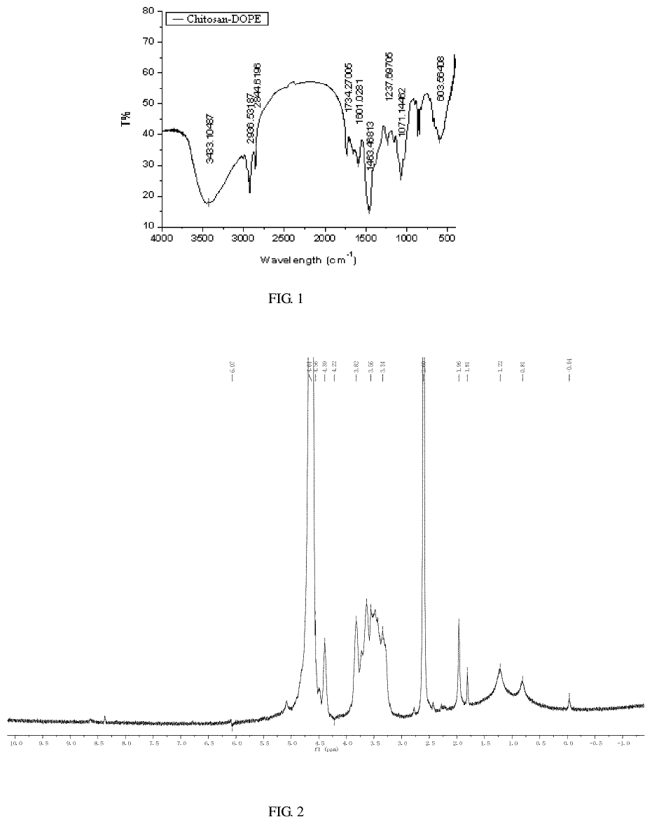

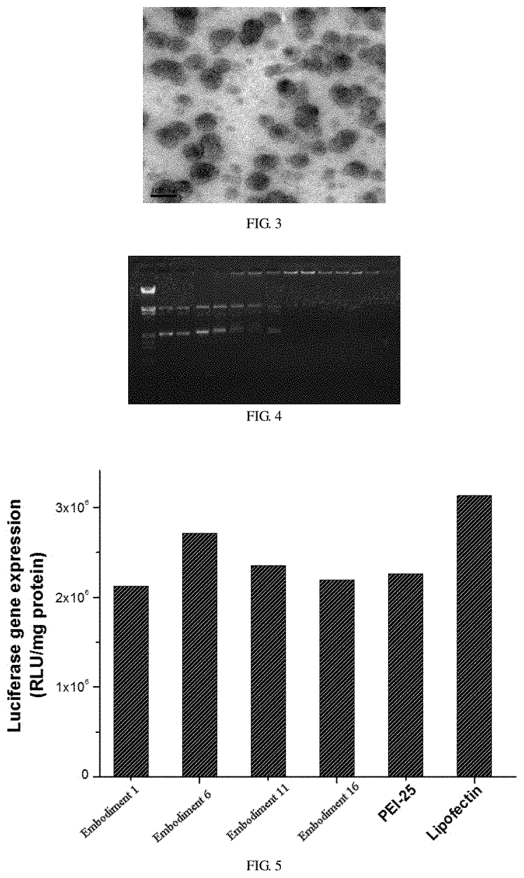

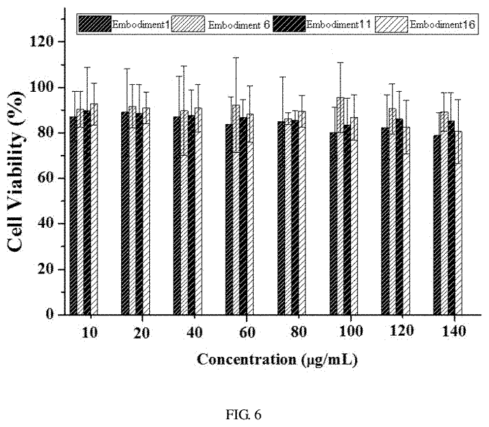

### DETAILED DESCRIPTION OF THE PREFERRED EMBODIMENTS

The present invention is described in details below with reference to the drawings and specific examples, but it is not intended to limit the scope of the invention. Unless otherwise stated, the experimental methods used in the present invention are all conventional methods, and the used experimental equipment, materials, reagents, and the like can be purchased from chemical companies.

### Example 1

0.5 g chitosan (CSO) having a molecular weight of 5 kDa is dissolved in 100 mL water, and sonicated for 30 min to dissolve it sufficiently. Under stirring, the aqueous solution of chitosan is dropwise added to a DMSO solution of dithiobissuccinimidyl propionate, after reacting at 30° C. for 2 h, 0.5 g an ethanol solution of 1,2-dioleoyl-sn-glycero-3-phosphoethanolamine is continuously added dropwise to the reaction solution, after reacting at 30° C. for 2 h, thereafter the reaction solution is subjected to rotary evaporation, dialysis and lyophilize to prepare redox-responsive 1,2-dioleoyl-sn-glycero-3-phosphoethanolamine chitosan.

1 mg/mL the redox-responsive 1,2-dioleoyl-sn-glycero-3-phosphoethanolamine chitosan aqueous solution is prepared, and take 100 μL to mix with 1 mL of DOTAP cationic liposome containing SPIO by ultrasonic method, then left stand for 1 h, and modifying the liposome by means of post-insertion and self-assembly to prepare a liposome drug carrier having a redox-responsive chitosan brush on a surface thereof.

### Example 2

0.5 g chitosan (CSO) having a molecular weight of 5 kDa is dissolved in 100 mL water, and sonicated for 30 min to dissolve it sufficiently. Under stirring, the aqueous solution of chitosan is dropwise added to a DMSO solution of dithiobissuccinimidyl propionate, after reacting at 30° C. for 2 h, 0.5 g an ethanol solution of 1,2-distearoyl-sn-glycero-3-phosphatidylethanolamine is continuously added dropwise to the reaction solution, reacting at 30° C. for 2 h, thereafter the reaction solution is subjected to rotary evaporation, dialysis and lyophilize to prepare redox-responsive 1,2-distearoyl-sn-glycero-3-phosphatidylethanolamine chitosan.

1 mg/mL the redox-responsive 1,2-distearoyl-sn-glycero-3-phosphatidylethanolamine chitosan aqueous solution is prepared, and take 100 μL to mix with 1 mL of DOTAP cationic liposome containing SPIO by ultrasonic method, then left stand for 1 h, and modifying the liposome by means of post-insertion and self-assembly to prepare a liposome drug carrier having a redox-responsive chitosan brush on a surface thereof.

### Example 3

0.5 g chitosan (CSO) having a molecular weight of 5 kDa is dissolved in 100 mL water, and sonicated for 30 min to dissolve it sufficiently. Under stirring, the aqueous solution of chitosan is dropwise added to a DMSO solution of dithiobissuccinimidyl propionate, after reacting at 30° C. for 2 h, 0.5 g an ethanol solution of 1,2-dilauroyl-sn-glycero-3-phosphoethanolamine is continuously added dropwise to the reaction solution, reacting at 30° C. for 2 h, thereafter the reaction solution is subjected to rotary evaporation, dialysis and lyophilize to prepare redox-responsive 1,2-dilauroyl-sn-glycero-3-phosphoethanolamine chitosan.

1 mg/mL the redox-responsive 1,2-dilauroyl-sn-glycero-3-phosphoethanolamine chitosan aqueous solution is prepared, and take 100 μL to mix with 1 mL of DOTAP cationic liposome containing SPIO by ultrasonic method, then left stand for 1 h, and modifying the liposome by means of post-insertion and self-assembly to prepare a liposome drug carrier having a redox-responsive chitosan brush on a surface thereof.

### Example 4

0.5 g chitosan (CSO) having a molecular weight of 1 kDa is dissolved in 100 mL water, and sonicated for 30 min to dissolved it sufficiently. Under stirring, the aqueous solution of chitosan is dropwise added to a DMSO solution of dithiobissuccinimidyl propionate, after reacting at 30° C. for 2 h, 0.5 g an ethanol solution of 1,2-dimyristoyl-sn-glycero-3-phosphatidylethanolamine is continuously added dropwise to the reaction solution, reacting at 30° C. for 2 h, thereafter the reaction solution is subjected to rotary evaporation, dialysis and lyophilize to prepare redox-responsive 1,2-dimyristoyl-sn-glycero-3-phosphatidylethanolamine chitosan.

1 mg/mL the redox-responsive 1,2-dimyristoyl-sn-glycero-3-phosphatidylethanolamine chitosan aqueous solution is prepared, and take 100 μL to mix with 1 mL of Lipofectin cationic liposome containing SPIO by ultrasonic method, then left stand for 1 h, and modifying the liposome by means of post-insertion and self-assembly to prepare a liposome drug carrier having a redox-responsive chitosan brush on a surface thereof.

### Example 5

0.5 g chitosan (CSO) having a molecular weight of 1 kDa is dissolved in 100 mL water, and sonicated for 30 min to dissolve it sufficiently. Under stirring, the aqueous solution of chitosan is dropwise added to a DMSO solution of dithiobissuccinimidyl propionate, after reacting at 30° C. for 2 h, 0.5 g an ethanol solution of 1,2-dipalmitoyl-sn-glycero-3-phosphoethanolamine is continuously added dropwise to the reaction solution, reacting at 30° C. for 2 h, thereafter the reaction solution is subjected to rotary evaporation, dialysis and lyophilize to prepare redox-responsive 1,2-dipalmitoyl-sn-glycero-3-phosphoethanolamine chitosan.

1 mg/mL the redox-responsive 1,2-dipalmitoyl-sn-glycero-3-phosphoethanolamine chitosan aqueous solution is prepared, and take 100 μL to mix with 1 mL of Lipofectin cationic liposome containing SPIO by ultrasonic method, then left stand for 1 h, and modifying the liposome by means of post-insertion and self-assembly to prepare a liposome drug carrier having a redox-responsive chitosan brush on a surface thereof.

### Example 6

0.5 g chitosan (CSO) having a molecular weight of 1 kDa is dissolved in 100 mL water, and sonicated for 30 min to dissolve it sufficiently. Under stirring, the aqueous solution of chitosan is dropwise added to a DMSO solution of dithiobissuccinimidyl propionate, after reacting at 30° C. for 2 h, 1.0 g an ethanol solution of 1,2-dioleoyl-sn-glycero-3-phosphoethanolamine is continuously added dropwise to the reaction solution, reacting at 30° C. for 2 h, thereafter the reaction solution is subjected to rotary evaporation, dialysis and lyophilize to prepare redox-responsive 1,2-dioleoyl-sn-glycero-3-phosphoethanolamine chitosan.

1 mg/mL the redox-responsive 1,2-dioleoyl-sn-glycero-3-phosphoethanolamine chitosan aqueous solution is prepared, and take 100 μL to mix with 1 mL of Lipofectamin™ 2000 cationic liposome containing SPIO by ultrasonic method, then left stand for 1 h, and modifying the liposome by means of post-insertion and self-assembly to prepare a liposome drug carrier having a redox-responsive chitosan brush on a surface thereof.

### Example 7

0.5 g chitosan (CSO) having a molecular weight of 5 kDa is dissolved in 100 mL water, and sonicated for 30 min to dissolve it sufficiently. Under stirring, the aqueous solution of chitosan is dropwise added to a DMSO solution of dithiobissuccinimidyl propionate, after reacting at 30° C. for 2 h, 1.0 g an ethanol solution of 1,2-distearoyl-sn-glycero-3-phosphatidylethanolamine is continuously added dropwise to the reaction solution, reacting at 30° C. for 2 h, thereafter the reaction solution is subjected to rotary evaporation, dialysis and lyophilize to prepare redox-responsive 1,2-distearoyl-sn-glycero-3-phosphatidylethanolamine chitosan.

1 mg/mL the redox-responsive 1,2-distearoyl-sn-glycero-3-phosphatidylethanolamine chitosan aqueous solution is prepared, and take 100 μL to mix with 1 mL of DOTAP cationic liposome containing SPIO by ultrasonic method, then left stand for 1 h, and modifying the liposome by means of post-insertion and self-assembly to prepare a liposome drug carrier having a redox-responsive chitosan brush on a surface thereof.

### Example 8

0.5 g chitosan (CSO) having a molecular weight of 1 kDa is dissolved in 100 mL water, and sonicated for 30 min to dissolve it sufficiently. Under stirring, the aqueous solution of chitosan is dropwise added to a DMSO solution of dithiobissuccinimidyl propionate, after reacting at 30° C. for 2 h, 1.0 g an ethanol solution of 1,2-dilauroyl-sn-glycero-3-phosphoethanolamine is continuously added dropwise to the reaction solution, reacting at 30° C. for 2 h, thereafter the reaction solution is subjected to rotary evaporation, dialysis and lyophilize to prepare redox-responsive 1,2-dilauroyl-sn-glycero-3-phosphoethanolamine chitosan.

1 mg/mL the redox-responsive 1,2-dilauroyl-sn-glycero-3-phosphoethanolamine chitosan aqueous solution is prepared, and take 100 μL to mix with 1 mL of Lipofectamin™ 2000 cationic liposome containing SPIO by ultrasonic method, then left stand for 1 h, and modifying the liposome by means of post-insertion and self-assembly to prepare a liposome drug carrier having a redox-responsive chitosan brush on a surface thereof.

### Example 9

0.5 g chitosan (CSO) having a molecular weight of 5 kDa is dissolved in 100 mL water, and sonicated for 30 min to dissolve it sufficiently. Under stirring, the aqueous solution of chitosan is dropwise added to a DMSO solution of dithiobissuccinimidyl propionate, after reacting at 30° C. for 2 h, 1.0 g an ethanol solution of 1,2-dimyristoyl-sn-glycero-3-phosphatidylethanolamine is continuously added dropwise to the reaction solution, reacting at 30° C. for 2 h, thereafter the reaction solution is subjected to rotary evaporation, dialysis and lyophilize to prepare redox-responsive 1,2-dimyristoyl-sn-glycero-3-phosphatidylethanolamine chitosan.

1 mg/mL the redox-responsive 1,2-dimyristoyl-sn-glycero-3-phosphatidylethanolamine chitosan aqueous solution is prepared, and take 100 μL to mix with 1 mL of DOTAP cationic liposome containing SPIO by ultrasonic method, then left stand for 1 h, and modifying the liposome by means of post-insertion and self-assembly to prepare a liposome drug carrier having a redox-responsive chitosan brush on a surface thereof.

### Example 10

0.5 g chitosan (CSO) having a molecular weight of 1 kDa is dissolved in 100 mL water, and sonicated for 30 min to dissolve it sufficiently. Under stirring, the aqueous solution of chitosan is dropwise added to a DMSO solution of dithiobis(succinimidyl propionate), after reacting at 30° C. for 2 h, 1.0 g an ethanol solution of 1,2-dipalmitoyl-sn-glycero-3-phosphoethanolamine is continuously added dropwise to the reaction solution, reacting at 30° C. for 2 h, thereafter the reaction solution is subjected to rotary evaporation, dialysis and lyophilize to prepare redox-responsive 1,2-dipalmitoyl-sn-glycero-3-phosphoethanolamine chitosan.

1 mg/mL the redox-responsive 1,2-dipalmitoyl-sn-glycero-3-phosphoethanolamine chitosan aqueous solution is prepared, and take 100 μL to mix with 1 mL of DOTAP cationic liposome containing SPIO by ultrasonic method, then left stand for 1 h, and modifying the liposome by means of post-insertion and self-assembly to prepare a liposome drug carrier having a redox-responsive chitosan brush on a surface thereof.

### Example 11

0.5 g chitosan (CSO) having a molecular weight of 1 kDa is dissolved in 100 mL water, and sonicated for 30 min to sufficiently dissolve it. Under stirring, the aqueous solution of chitosan is dropwise added to a DMSO solution of dithiobis(succinimidyl propionate), after reacting at 30° C. for 4 h, 1.0 g an ethanol solution of 1,2-dioleoyl-sn-glycero-3-phosphoethanolamine is continuously added dropwise to the reaction solution, reacting at 30° C. for 4 h, thereafter the reaction solution is subjected to rotary evaporation, dialysis and lyophilize to prepare redox-responsive 1,2-dioleoyl-sn-glycero-3-phosphoethanolamine chitosan.

1 mg/mL the redox-responsive 1,2-dioleoyl-sn-glycero-3-phosphoethanolamine chitosan aqueous solution is prepared, and take 500 μL to mix with 1 mL of DOTAP cationic liposome containing SPIO by ultrasonic method, then left stand for 1 h, and modifying the liposome by means of post-insertion and self-assembly to prepare a liposome drug carrier having a redox-responsive chitosan brush on a surface thereof.

### Example 12

0.5 g chitosan (CSO) having a molecular weight of 5 kDa is dissolved in 100 mL water, and sonicated for 30 min to sufficiently dissolve it. Under stirring, the aqueous solution of chitosan is dropwise added to a DMSO solution of dithiobis(succinimidyl propionate), after reacting at 30° C. for 4 h, 1.0 g an ethanol solution of 1,2-distearoyl-sn-glycero-3-phosphatidylethanolamine is continuously added dropwise to the reaction solution, reacting at 30° C. for 4 h, thereafter the reaction solution is subjected to rotary evaporation, dialysis and lyophilize to prepare redox-responsive 1,2-distearoyl-sn-glycero-3-phosphatidylethanolamine chitosan.

1 mg/mL the redox-responsive 1,2-distearoyl-sn-glycero-3-phosphatidylethanolamine chitosan aqueous solution is prepared, and take 500 μL to mix with 1 mL of DOTAP cationic liposome containing SPIO by ultrasonic method, then left stand for 1 h, and modifying the liposome by means of post-insertion and self-assembly to prepare a liposome drug carrier having a redox-responsive chitosan brush on a surface thereof.

### Example 13

0.5 g chitosan (CSO) having a molecular weight of 5 kDa is dissolved in 100 mL water, and sonicated for 30 min to sufficiently dissolve it. Under stirring, the aqueous solution of chitosan is dropwise added to a DMSO solution of dithiobissuccinimidyl propionate, after reacting at 30° C. for 4 h, 1.0 g an ethanol solution of 1,2-dilauroyl-sn-glycero-3-phosphoethanolamine is continuously added dropwise to the reaction solution, reacting at 30° C. for 4 h, thereafter the reaction solution is subjected to rotary evaporation, dialysis and lyophilize to prepare redox-responsive 1,2-dilauroyl-sn-glycero-3-phosphoethanolamine chitosan.

1 mg/mL the redox-responsive 1,2-dilauroyl-sn-glycero-3-phosphoethanolamine chitosan aqueous solution is prepared, and take 500 μL to mix with 1 mL of DOTAP cationic liposome containing SPIO by ultrasonic method, then left stand for 1 h, and modifying the liposome by means of post-insertion and self-assembly to prepare a liposome drug carrier having a redox-responsive chitosan brush on a surface thereof.

### Example 14

0.5 g chitosan (CSO) having a molecular weight of 5 kDa is dissolved in 100 mL water, and sonicated for 30 min to sufficiently dissolve it. Under stirring, the aqueous solution of chitosan is dropwise added to a DMSO solution of dithiobissuccinimidyl propionate, after reacting at 30° C. for 4 h, 1.0 g an ethanol solution of 1,2-dimyristoyl-sn-glycero-3-phosphatidylethanolamine is continuously added dropwise to the reaction solution, reacting at 30° C. for 4 h, thereafter the reaction solution is subjected to rotary evaporation, dialysis and lyophilize to prepare redox-responsive 1,2-dimyristoyl-sn-glycero-3-phosphatidylethanolamine chitosan.

1 mg/mL the redox-responsive 1,2-dimyristoyl-sn-glycero-3-phosphatidylethanolamine chitosan aqueous solution is prepared, and take 500 μL to mix with 1 mL of DOTAP cationic liposome containing SPIO by ultrasonic method, then left stand for 1 h, and modifying the liposome by means of post-insertion and self-assembly to prepare a liposome drug carrier having a redox-responsive chitosan brush on a surface thereof.

### Example 15

0.5 g chitosan (CSO) having a molecular weight of 1 kDa is dissolved in 100 mL water, and sonicated for 30 min to sufficiently dissolve it. Under stirring, the aqueous solution of chitosan is dropwise added to a DMSO solution of dithiobissuccinimidyl propionate, after reacting at 30° C. for 4 h, 1.0 g an ethanol solution of 1,2-dipalmitoyl-sn-glycero-3-phosphoethanolamine is continuously added dropwise to the reaction solution, reacting at 30° C. for 4 h, thereafter the reaction solution is subjected to rotary evaporation, dialysis and lyophilize to prepare redox-responsive 1,2-dipalmitoyl-sn-glycero-3-phosphoethanolamine chitosan.

1 mg/mL the redox-responsive 1,2-dipalmitoyl-sn-glycero-3-phosphoethanolamine chitosan aqueous solution is prepared, and take 500 μL to mix with 1 mL of DOTAP cationic liposome containing SPIO by ultrasonic method, then left stand for 1 h, and modifying the liposome by means of post-insertion and self-assembly to prepare a liposome drug carrier having a redox-responsive chitosan brush on a surface thereof.

### Example 16

0.5 g chitosan (CSO) having a molecular weight of 5 kDa is dissolved in 100 mL water, and sonicated for 30 min to sufficiently dissolve it. Under stirring, the aqueous solution of chitosan is dropwise added to a DMSO solution of dithiobissuccinimidyl propionate, after reacting at 40° C. for 4 h, 1.0 g an ethanol solution of 1,2-dioleoyl-sn-glycero-3-phosphoethanolamine is continuously added dropwise to the reaction solution, reacting at 40° C. for 4 h, thereafter the reaction solution is subjected to rotary evaporation, dialysis and lyophilize to prepare redox-responsive 1,2-dioleoyl-sn-glycero-3-phosphoethanolamine chitosan.

1 mg/mL the redox-responsive 1,2-dioleoyl-sn-glycero-3-phosphoethanolamine chitosan aqueous solution is prepared, and take 500 μL to mix with 1 mL of DOTAP cationic liposome containing SPIO by ultrasonic method, then left stand for 2 h, and modifying the liposome by means of post-insertion and self-assembly to prepare a liposome drug carrier having a redox-responsive chitosan brush on a surface thereof.

### Example 17

0.5 g chitosan (CSO) having a molecular weight of 1 kDa is dissolved in 100 mL water, and sonicated for 30 min to sufficiently dissolve it. Under stirring, the aqueous solution of chitosan is dropwise added to a DMSO solution of dithiobissuccinimidyl propionate, after reacting at 40° C. for 4 h, 1.0 g an ethanol solution of 1,2-distearoyl-sn-glycero-3-phosphatidylethanolamine is continuously added dropwise to the reaction solution, reacting at 40° C. for 4 h, thereafter the reaction solution is subjected to rotary evaporation, dialysis and lyophilize to prepare redox-responsive 1,2-distearoyl-sn-glycero-3-phosphatidylethanolamine chitosan.

1 mg/mL the redox-responsive 1,2-distearoyl-sn-glycero-3-phosphatidylethanolamine chitosan aqueous solution is prepared, and take 500 μL to mix with 1 mL of DOTAP cationic liposome containing SPIO by ultrasonic method, then left stand for 2 h, and modifying the liposome by means of post-insertion and self-assembly to prepare a liposome drug carrier having a redox-responsive chitosan brush on a surface thereof.

### Example 18

0.5 g chitosan (CSO) having a molecular weight of 1 kDa is dissolved in 100 mL water, and sonicated for 30 min to sufficiently dissolve it. Under stirring, the aqueous solution of chitosan is dropwise added to a DMSO solution of dithiobissuccinimidyl propionate, after reacting at 40° C. for 4 h, 1.0 g an ethanol solution of 1,2-dilauroyl-sn-glycero-3-phosphoethanolamine is continuously added dropwise to the reaction solution, reacting at 40° C. for 4 h, thereafter the reaction solution is subjected to rotary evaporation, dialysis and lyophilize to prepare redox-responsive 1,2-dilauroyl-sn-glycero-3-phosphoethanolamine chitosan.

1 mg/mL the redox-responsive 1,2-dilauroyl-sn-glycero-3-phosphoethanolamine chitosan aqueous solution is prepared, and take 500 μL to mix with 1 mL of DOTAP cationic liposome containing SPIO by ultrasonic method, then left stand for 2 h, and modifying the liposome by means of post-insertion and self-assembly to prepare a liposome drug carrier having a redox-responsive chitosan brush on a surface thereof.

### Example 19

0.5 g chitosan (CSO) having a molecular weight of 1 kDa is dissolved in 100 mL water, and sonicated for 30 min to sufficiently dissolve it. Under stirring, the aqueous solution of chitosan is dropwise added to a DMSO solution of dithiobis(succinimidyl propionate), after reacting at 40° C. for 4 h, 1.0 g an ethanol solution of 1,2-dimyristoyl-sn-glycero-3-phosphatidylethanolamine is continuously added dropwise to the reaction solution, reacting at 40° C. for 4 h, thereafter the reaction solution is subjected to rotary evaporation, dialysis and lyophilize to prepare redox-responsive 1,2-dimyristoyl-sn-glycero-3-phosphatidylethanolamine chitosan.

1 mg/mL the redox-responsive 1,2-dimyristoyl-sn-glycero-3-phosphatidylethanolamine chitosan aqueous solution is prepared, and take 500 μL to mix with 1 mL of DOTAP cationic liposome containing SPIO by ultrasonic method, then left stand for 2 h, and modifying the liposome by means of post-insertion and self-assembly to prepare a liposome drug carrier having a redox-responsive chitosan brush on a surface thereof.

### Example 20

0.5 g chitosan (CSO) having a molecular weight of 5 kDa is dissolved in 100 mL water, and sonicated for 30 min to sufficiently dissolve it. Under stirring, the aqueous solution of chitosan is dropwise added to a DMSO solution of dithiobissuccinimidyl propionate, after reacting at 40° C. for 4 h, 1.0 g an ethanol solution of 1,2-dipalmitoyl-sn-glycero-3-phosphoethanolamine is continuously added dropwise to the reaction solution, reacting at 40° C. for 4 h, thereafter the reaction solution is subjected to rotary evaporation, dialysis and lyophilize to prepare redox-responsive 1,2-dipalmitoyl-sn-glycero-3-phosphoethanolamine chitosan.

1 mg/mL the redox-responsive 1,2-dipalmitoyl-sn-glycero-3-phosphoethanolamine chitosan aqueous solution is prepared, and take 500 μL to mix with 1 mL of DOTAP cationic liposome containing SPIO by ultrasonic method, then left stand for 2 h, and modifying the liposome by means of post-insertion and self-assembly to prepare a liposome drug carrier having a redox-responsive chitosan brush on a surface thereof.

Gene Transfer Assay of Liposome Having a Redox-Responsive Chitosan Brush.

A pGL3 plasmid is used as a reporter gene to evaluate gene transfer properties of the liposome carrier having a redox-responsive chitosan brush, and the cells used are human non-small cell lung cancer A549 cell lines. The cultured cells are plated, cultured in an incubator until the cell fusion degree reaches 80%, and then subject to gene transfer. During transporting, the complete medium is aspirated, and washed twice with PBS, and transported under serum conditions, 400 μL of medium containing 10% serum and different N/P ratios (mass ratio) of the redox-responsive polysome@SPIO (Example 3)—DNA complexes are added, after culture for 18 h, the medium is aspirated, and after replacing with a fresh medium containing 10% serum and continually culture for 32 h. The photon intensity is measured by a BioTek Synergy2 multi-function microplate reader according to the instruction book provided by the luciferase kit, and the total protein concentration is detected by BCA, thereby normalizing the results to RLU/mg protein (relative photon number per mg of protein).

Cytotoxicity of Liposome Having a Redox-Responsive Chitosan Brush.

The cytotoxicity of the carrier is evaluated by MTT method. Cells are grown in a 96-well cell culture plate, 3 wells in parallel, 5×10⁴ cells were grown per well, and cultured at 37° C. in a 5% CO₂ incubator until the cell fusion degree reaches 85% or more. The medium is removed, PBS is used to wash twice, a fresh medium and the carrier to be measured are added, after culturing for 24 h, 20 μL 5 mg/mL of MTT solution is added to each well, continuously culturing at 37° C. for 4 h, the medium is removed, and the culture is terminated. Succinate dehydrogenase in mitochondria of living cells reduces MTT to generate formazan, which is dissolved by adding 150 μL DMSO to each well, and continually incubating at 37° C. for 30 min. The absorbance value of each well at 570 nm was measured by a multi-functional microplate reader (Sunrise Tecan), the 96-well plate was automatically mixed for 600 s before detection, and a cell-free medium being used to zero the microplate reader. Cell viability is calculated according Formula 1.1:

cell viability (%)=A570_{SMP}/A570_{CTL}×100 (1.1)

Wherein A570_{SMP} refers to an absorbance value of a cell plate to which the carrier or complex to be measured is added, and A570_{CTL} refers to an absorbance value of a cell plate containing only the medium.

The above is merely the preferred examples of the present invention, and is not intended to limit the present invention. Any modifications, equivalent substitutions, improvements, and the like that made within the spirit and principles of the present invention should be included in the protection of the present invention.

What is claimed is:

1. A redox-responsive chitosan, wherein the chitosan has a structure of formula (I): 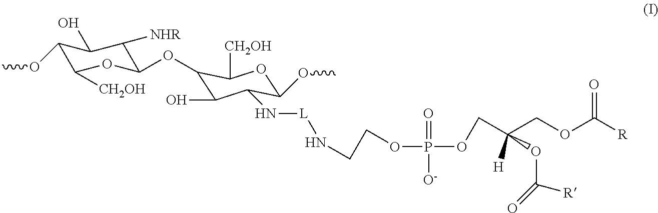
wherein, L=—CO—(CH₂)ₐ—S—S—(CH₂)_{b}—CO—, a=1-5, b=1-5; and
R and R′ are identical or different CₓH_{y}, wherein x=11-17, y=21-35.

2. The redox-responsive chitosan according to claim 1, wherein L=—CO—(CH₂)₂—S—S—(CH₂)₂—CO—, R and R′ are identical or different C₁₁H₂₃, C₁₃H₂₇, C₁₇H₃₅ or C₁₇H₃₃.

3. A method of preparing the redox-responsive chitosan according to claim 1, wherein firstly the chitosan is dissolved in water, sufficiently dissolved, under stirring, dropwise added to a DMSO solution of dithiobis succinimidyl-substituted ester, after reacting at 20-60° C. for 1-24 h, an ethanol solution of double fatty chain substituent phosphatidylethanolamine is continuously added dropwise to the reaction solution, reacting at 20-60° C. for 1-24 h, after rotary evaporation, the reaction solution is subjected to dialysis, lyophilize, to prepare the redox-responsive chitosan.

4. The method of preparing the redox-responsive chitosan according to claim 3, wherein a weight average molecular weight of the chitosan is 500-10000 Da, and a degree of deacetylation is 65-95%.

5. The method of preparing the redox-responsive chitosan according to claim 3, wherein the double fatty chain substituent phosphatidylethanolamine is one or more of 1,2-dilauroyl-sn-glycero-3-phosphoethanolamine, 1,2-distearoyl-sn-glycero-3-phosphatidylethanolamine, 1,2-dimyristoyl-sn-glycero-3-phosphatidylethanolamine, 1,2-dipalmitoyl-sn-glycero-3-phosphoethanolamine, and 1,2-dioleoyl-sn-glycero-3-phosphoethanolamine.

6. The method of preparing the redox-responsive chitosan according to claim 3, wherein the double fatty chain substituent phosphatidylethanolamine is used in an amount of 0.1-1 times a molar equivalent of a repeating unit of the chitosan, and a reaction condition is stirring and reacting at 20-50° C. for 2-48 h.

7. The method of preparing the redox-responsive chitosan according to claim 3, wherein the double fatty chain substituent phosphatidylethanolamine is used in an amount of 0.3-0.6 times a molar equivalent of a repeating unit of the chitosan, and a reaction condition is stirring and reacting at 30-50° C. for 4-12 h.

8. A method of preparing a redox-responsive chitosan-liposome, wherein using the double fatty chain substituent phosphatidylethanolamine according to claim 1 to modify a cationic liposome by a post-insertion and self-assembly method, to prepare the redox-responsive chitosan-liposome.

9. The method of preparing the redox-responsive chitosan-liposome according to claim 8, wherein the cationic liposome is any one of DOTAP, Lipofectin and Lipofectamin™ 2000, and a hydrophilic core of the cationic liposome encapsulating super-paramagnetic ferroferric oxide nanoparticles having a particle diameter of 1-30 nm.

10. Use of the redox-responsive chitosan-liposome according to claim 8 in drug delivery.
